(19) United States
(12) Patent Application Publication
(10) Pub. No.: US 2013/0322713 A1
(43) Pub. Date: Dec. 5, 2013

(54) Color map design method for assessment of the deviation from established normal population statistics and its application to quantitative medical images

(71) Applicant: Isis Innovation Ltd., Oxford (GB)

(72) Inventors: Stefan K. Piechnik, Oxford (GB); Matthew D. Robson, Oxford (GB)

(21) Appl. No.: 13/986,716

(22) Filed: May 28, 2013

Related U.S. Application Data
(60) Provisional application No. 61/689,067, filed on May 29, 2012.

(51) Int. Cl. A61B 5/055; G01R 33/56; A61B 6/00; A61B 8/08; A61B 8/13; A61B 6/03

(52) U.S. Cl. CPC A61B 5/055; A61B 8/13; A61B 6/032; A61B 6/52; A61B 8/52; G01R 33/56
USPC 382/128

(57) ABSTRACT

Systems and methods for providing color maps for use in medical imaging are described for assessment of the deviation from established normal population statistics and application to quantitative medical imaging. Statistical descriptors are used to determine the color lookup table to be applied to the medical image in order to create a meaningful color map, in particular dedicated to assess the extent of deviation from the normal range.

### CROSS REFERENCE TO RELATED APPLICATIONS

This application claims priority to co-pending U.S. provisional application entitled “COLOR MAP DESIGN METHOD FOR ASSESSMENT OF THE DEVIATION FROM ESTABLISHED NORMAL POPULATION STATISTICS AND ITS APPLICATION TO QUANTITATIVE MEDICAL IMAGES” having Ser. No. 61/689,067, filed May 29, 2012, which is hereby incorporated by reference in its entirety.

### TECHNICAL FIELD

The present disclosure generally relates to medical imaging and more particularly, relates to systems and methods for providing a color map design for assessment, that can be direct and immediate, of the deviation from established normal population statistics and its application to quantitative medical imaging, such as but not limited to cardiovascular T1 mapping methods and images.

### BACKGROUND

Until now, arbitrary and usually meaningless color schemes were used to describe quantitative maps. Typically “hotter” colors like white, red or yellow were used to represent higher values and “colder” selections like black, blue or magenta were assigned to low value ranges. The freedom in map selection was large and depended on arbitrary choice for the scale and ranges used.

As prior work there is research and there are patents for security scanning based on X-ray imaging. Based on X-ray opacity there have been schemes proposed to distinguish several types of image density as separate colors with a goal to make them separable from each other as to define clear differences to the human eye for metal, organic matter, fluids, etc. The selection is relevant to security scanning based on the measurement of best way to distinguish object types. No prior work has been directed to presenting a formal definition of color maps from population data statistics to be dedicated to distinguish the normal values from abnormal range in order to guide on-the-spot clinical evaluation and quantitative medical imaging.

Accordingly, there is a need to address the aforementioned deficiencies and inadequacies.

### SUMMARY

Briefly described, one embodiment, among others, is a method for providing color maps for use in medical imaging, and in particular for providing a color map design for assessment of the deviation from established normal population statistics and application to quantitative medical imaging. In an embodiment, methods and systems are described for providing a color map for medical imaging, comprising the steps of:
(a) acquiring a medical image;
(b) generating a color lookup table for representation of the medical image by:
   (i) assigning a specific color to represent an established normal range of values in the image using one or more statistical descriptors of respective data distributions,
   (ii) assigning the extent of deviation from the normal color or from an accepted threshold using a statistical descriptor to distinguish an altered state (such as disease or intervention state). This can include, but is not limited to, use of respective standard deviations, standard errors, percentile ranges or detection thresholds for desired sensitivity or specificity of the respective altered state detection.
   (iii) using colors different than the color assigned in step (i), for example accepted contrasting colors, to represent targets for the deviation from the normal range of values or from accepted thresholds (using, for example, the descriptor(s) listed in step ii)) or additional normal ranges for one or more additional types of tissues (assigned as in step (i)), and
   (iv) setting color transitions applying the respective statistical descriptors as in step (ii); and
(c) applying the generated color lookup table for the color transitions to the medical image.

Suitable statistical descriptors that can be used include, but are not limited to, mean (average) value, standard deviation (SD), standard error of mean (SEM), Confidence ranges, median and/or percentile ranges with any respective sums and multiplications. The statistics can be calculated from prior studies or they can be calculated for each dataset separately by selecting adequate reference areas. Other formal statistical descriptors that can be determined with respect to established differences from abnormal conditions can also be used, e.g., sensitivity or specificity based thresholds.

The present systems and methods can be applied to any quantitative medical imaging, including, for example, magnetic resonance (MR) imaging (including but not limited to T1 mapping), ultrasound or computed tomography (CT) to replace traditional grayscale windows with normalized color scales.

Other systems, methods, features, and advantages of the present disclosure for providing a color map design for immediate assessment of the deviation from established normal population statistics and its application to cardiovascular T1 mapping methods and images will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 depicts a flow chart for the present systems and methods for providing a color map design for assessment of the deviation from established normal population statistics and its application to quantitative medical imaging, for example cardiovascular T1 mapping methods and images.

FIG. 2 shows one embodiment of the present systems and methods in which derivations from a gaussian formula are used to determine smooth transitions between colors. In this embodiment the low values are indicated in blue and high in red hue. Approaching the limits of color lookup table (and/or of possible values range) are indicated by progressive reduction of image intensity (darkening).

FIG. 3 shows another embodiment of the present systems and methods in which a piece-wise linear algorithm is used to determine transitions in a fashion that is symmetric around the green (value of 75). Additional points indicate transitions towards the ends of the color lookup table.

FIG. 4 depicts a further embodiment of the present systems and methods with an asymmetric appearance of more pronounced upwards transition from normal range and diminished sensitivity to downwards transition.

FIG. 5 depicts additional examples of the present systems and methods for presenting color mapping to identify suspected pathology (or artifacts) as departure from normal range.

FIG. 6 depicts a flow chart for one embodiment for providing color mapping for immediate assessment of deviation from normal range of the present systems and methods.

### DETAILED DESCRIPTION

Having summarized various aspects of the present disclosure, reference will now be made in detail to the description of the disclosure as illustrated in the drawings. While the disclosure will be described in connection with these drawings, there is no intent to limit it to the embodiment or embodiments disclosed herein. On the contrary, the intent is to cover all alternatives, modifications and equivalents included within the spirit and scope of the disclosure as defined by the appended claims.

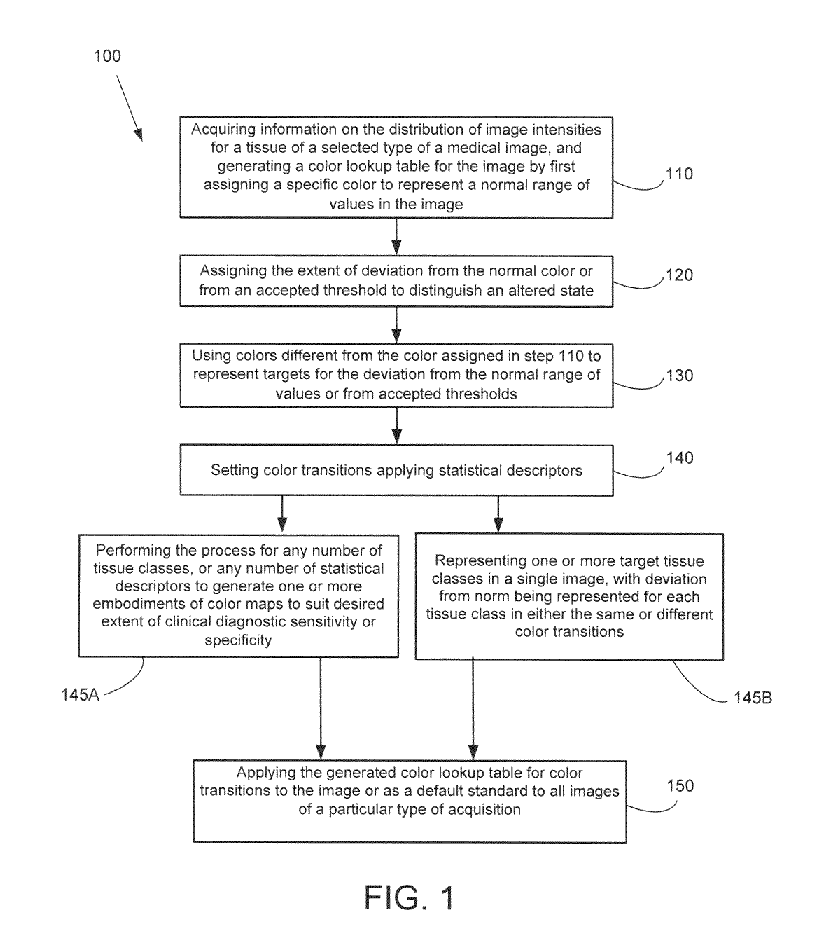

One embodiment of the present systems and methods is illustrated in FIG. 1. FIG. 1 depicts a flow chart 100 for providing a color map design for assessment of the deviation from established normal population statistics and its application to quantitative medical imaging, such as cardiovascular T1 mapping methods and images. In a preferred embodiment the assessment is direct and immediate. In this embodiment a medical imaging device is provided, and a subject or patient is positioned in association with the imaging device. The imaging device is used to acquire one or more images. The acquisition may be of sufficient information on the distribution of image intensities for a particular tissue of a selected type of a medical image or images in a group of different subjects. The subjects may include patients, in particular clinical patients.

Upon acquisition, a color lookup table is generated for application to the image and representation of the image. For generating the color lookup table, first a specific color may be assigned 110 to represent an established normal range or ranges of values in the image. As an example, the color green can be selected to represent a normal range due to universal acceptance of this color as an “OK” sign or as a “Go” signal. The color green does not have to be selected, however. Another color or other colors can be selected by the user to represent a normal range. In one embodiment, the normal range is determined by using a statistical basis, for example by using one or more statistical descriptors of respective data distributions. This may include, various confidence intervals, midpoints and other ratios of the distance between average for separate tissue classes as appropriate.

The extent of deviation from the normal color or from an accepted threshold is assigned 120 using a statistical descriptor to distinguish an altered state (for example a disease or intervention state). The statistical descriptor can include, but is not limited to standard deviations, standard errors, percentile ranges or detection thresholds for desired sensitivity or specificity of the respective altered state detection.

Colors different than the color assigned to represent the normal range or ranges of values can be used 130 to represent targets for the deviation from the established normal range or ranges or from an accepted threshold. For example, specific color ranges as near range deviation towards high or low hues can be used. As an example, where the color green is selected to represent a normal range, then the colors red and blue can be selected as targets for near range deviation towards high or low hues, respectively, There may be more thresholds exemplified here as a change in color due to reaching low and high range possible of estimates.

Color transitions between the above described thresholds can be set 140 applying the respective statistical descriptors. In one embodiment this can be exemplified, for example, on broken line calculation of red−green−blue (RGB) saturations. In another embodiment this can be performed using other nonlinear functions and other color models such as a cyan-magenta-yellow-key (black) (CMYK) color model. For example, the exemplification can be performed by additional varying brightness. All parameters influencing the hue, the saturation and brightness, however, can be manipulated or kept stable if desired.

Contrary to previous color mapping techniques, color/hue/brightness, etc., transitions are not arbitrary but are set 140 to established statistical descriptors for the distribution of normal values. Examples of statistical descriptors that can be used include mean value, and standard deviation (SD) and its sums and multiplications. Other formal statistical descriptors that can be determined with respect to established differences from abnormal conditions can also be used, e.g., sensitivity or specificity based thresholds.

The above process may be performed 145A for any number of tissue classes, or any number of statistical descriptors to generate one or more embodiments of color maps to suit any desired extent of clinical diagnostic sensitivity of specificity. Non-limiting examples of tissue classes include myocardium liver, spleen, fat, blood pools, etc. The tissue classes can include any tissue class typically the subject of medical imaging, including for example tissues of any one or more internal organs of a body. One or more target tissue classes may be represented 145B this way in a single image, with deviation from norm being represented with either the same or different color transitions for each to be interpreted together within an anatomical location.

We can apply 150 the generated color lookup table for the desired color transitions to the selected image, in particular a medical image, or all images characterized by similar type of image qualities. An example of such is magnetic resonance (MR) imaging T1 mapping. The generated color look up table may also be applied as a default standard to all the medical images of a particular type of acquisition. The application of the present systems and methods, however, is not limited to T1 mapping or even more generally to MR imaging. They can be applied to any quantitative medical imaging acquisition, including, for example, ultrasound or computed tomography (CT) to replace traditional grayscale windows with normalized color scales. Moreover, the present systems and methods can be designed to allow the user to adjust the degree of transition either interactively to suit the user's own definition of the deviation from norm (scaling the color distribution around “normal” range) or in preset rigid intervals using evidence specific to various organs or diseases.

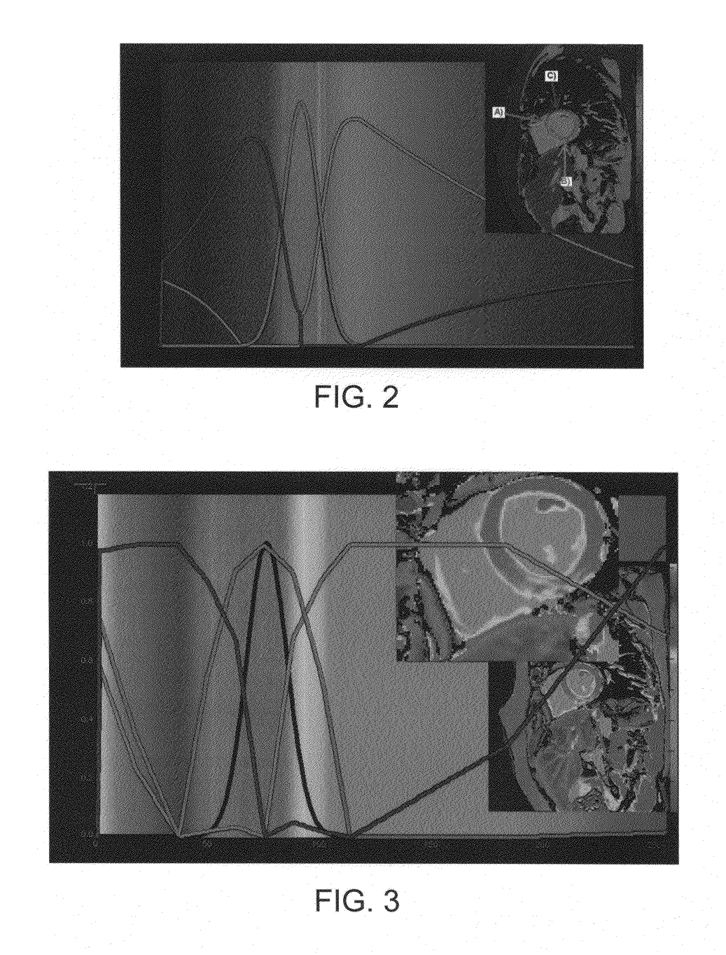

FIG. 2 depicts an embodiment of the present systems and methods in which a mean value statistical descriptor, in particular a gaussian formula based algorithm, is used to determine the transitions applied. In FIG. 2, RGB channel intensity is depicted on the Y axis. The X axis depicts color index from minimum to maximum value. The green channel is described as Gaussian corresponding to the distribution of normal values of myocardial relaxation times established in a small internal sample of subjects. The majority of the heart (Arrow A) is normal “green”. The lower part of the heart has visible reddish hue indicating a departure from normal to abnormally high T1 values (Arrow B). Departure towards low values outside norm is indicated as the blue streak (arrow C), likely due to image artifact. Extreme ranges are indicated by change in both luminosity (darkening) and hue (towards magenta).

FIG. 3 depicts a further embodiment applying the present systems and methods to T1 mapping. In this embodiment a different transition model is used to highlight the abnormal transition range than used in connection with FIG. 2, namely a linear-based algorithm. The color brightness has been optimized for consistency over the whole range of values (i.e sum of red+blue+green to be constant) except for very low values, where insertion of one or more arbitrary colors may indicate unfeasible values or error status (in here depicted as “black” color). In particular a symmetric function towards blue is used for the transitions in Green channel, but asymmetric functions for Red and Blue to address the possibility of definition of other quantitive transitions (herein dedicated to the range of blood (red) and liver (blue) T1 values), that have been set herein arbitrary fashion only for demonstration. Shallow changes in green hues for mean±SD with progressively steep changes for ±2*SD and 3±SD ranges demonstrate the use of a statistical descriptor to define transitions. Note the map luminosity has been normalised (total (R+G+B channels=100%) and the transition towards extremes have been added (magenta as max=250) and light blue towards 0). In this embodiment, the green normal range is wider than in FIG. 2, along with the transitional areas from green to red (i.e., the yellow portion) and from green to blue.

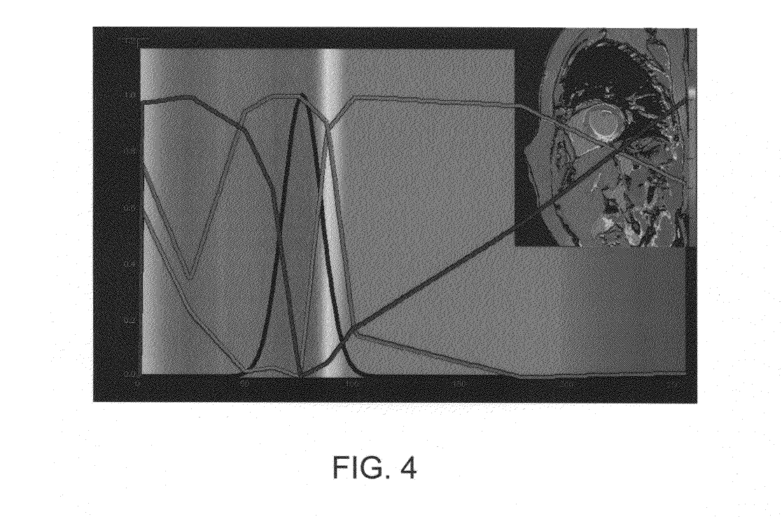

FIG. 4 depicts use of standard deviation (SD) as the statistical descriptor for the distribution of normal values. In an aspect, FIG. 4 depicts a piece-wise linear interpolated model with flat range in Green channel for T1 values within 0 to −1 standard deviation (SD) from average T1 to signify a method for marking presumed lack of interest in the green hue change in this range of normal validation of values. Other channels may be either set constant or change the hue as in this embodiment. The extremes are as before in FIG. 3. This exemplification makes the appearance of the lesion in the image brighter and as such exemplifies an embodiment of the present method that is dedicated to be more sensitive than specific to the abnormality, asymmetrically at the high range of values.

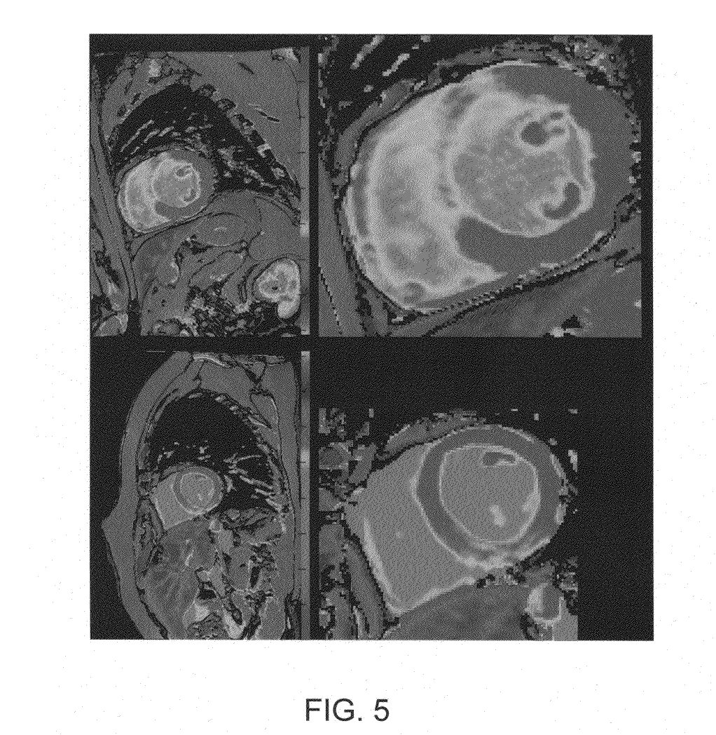

Additional examples of the use of the present color map to identify suspected pathology (or artifacts) as departure from normal “green” color range are depicted in FIG. 5. The left panels show whole images as obtained by the scanner with a calibration bar at the right side of each image. These images show clear distinction between colors for the normal heart tissue (green) contrasted with tissues characterised by lower T1 (blue, liver and fat) and long T1 values (blood, fluids in the stomach and kidneys) which provides a direct and immediate classification of the type of tissue appearing in the image. The right panels are zoomed in around the heart to emphasis the pathological appearance of the myocardium following the infarction (top-right panel 8-11 hours, and bottom-right panel 5-7 hours). The small area at the top-right panel at 4-o′clock shows blue hue that likely indicates an artifact, but may also indicate pathological changes such as infiltration of myocardium with fatty tissue.

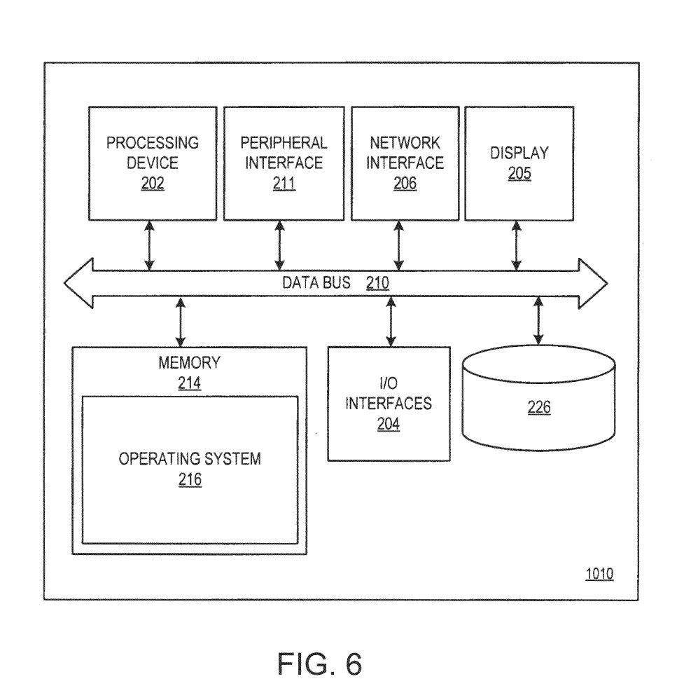

Reference is now made to FIG. 6, which depicts a system or apparatus 1010 in which the present method for providing a color map design for immediate assessment of the deviation from established normal population statistics and its application to quantitative medical imaging, for example cardiovascular T1 mapping methods and images, described herein may be implemented. In one or more aspects our present method may be carried out by programming logic executed in a computing environment, such as described herein.

The apparatus 1010 may be embodied in any one of a wide variety of wired and/or wireless computing devices, multiprocessor computing device, and so forth. As shown in FIG. 6, the apparatus 1010 comprises memory 214, a processing device 202, a number of input/output interfaces 204, a network interface 206, a display 205, a peripheral interface 211, and mass storage 226, wherein each of these devices are connected across a local data bus 210. The apparatus 1010 may be coupled to one or more peripheral measurement devices (not shown) connected to the apparatus 1010 via the peripheral interface 211.

The processing device 202 may include any custom made or commercially available processor, a central processing unit (CPU) or an auxiliary processor among several processors associated with the apparatus 1010, a semiconductor based microprocessor (in the form of a microchip), a macro-processor, one or more application specific integrated circuits (ASICs), a plurality of suitably configured digital logic gates, and other well-known electrical configurations comprising discrete elements both individually and in various combinations to coordinate the overall operation of the computing system.

The apparatus 1010 may comprise, for example, a hand-held device, a portable device, a computer, server, dedicated processing system, or other system, as can be appreciated. The hand-held device can be, for example, a smart mobile phone or a tablet. The computing environment of such device may include various input devices such as a keyboard, microphone, mouse, touch screen, or other device, as can be appreciated. By way of example, the system can comprise a stand-alone device or part of a network, such as a local area network (LAN), GPRS cellular network or wide area network (WAN).

The memory 214 can include any one of a combination of volatile memory elements (e.g., random-access memory (RAM, such as DRAM, MRAM and SRAM, etc.)), nonvolatile memory elements (e.g., ROM, hard drive, CDROM, etc.) and data storage components. Volatile components are those that do not retain data values upon loss of power. Nonvolatile components are those that retain data upon a loss of power. Thus, the memory 214 may also comprise, for example, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. The ROM may comprise, for example, a programmable read-only memory (PROM), an erasable programmable read-only memory (EPROM), an electrically erasable programmable read-only memory (EEPROM), or other like memory device.

The memory 214 typically comprises a native operating system 216, one or more native applications, emulation systems, or emulated applications for any of a variety of operating systems and/or emulated hardware platforms, emulated operating systems, etc. For example, the applications may include application specific software which may be configured to perform some or all of the color mapping technique described herein. In accordance with such embodiments, the application specific software is stored in memory 214 and executed by the processing device 202. One of ordinary skill in the art will appreciate that the memory 214 can, and typically will, comprise other components which have been omitted for purposes of brevity.

One or more input/output interfaces 204 provide any number of interfaces for the input and output of data. For example, where the apparatus 1010 comprises a personal computer, these components may interface with one or more user input devices 204. The input/output interfaces 204 may comprise the components with which a user interacts with the apparatus and therefore may comprise, for example, a keyboard, mouse, and a display, such as a liquid crystal display (LCD) monitor. The input/output interfaces 204 may also comprise, for example, a touch screen that serves both input and output functions. The display 205 may comprise a computer monitor, a plasma screen for a PC, a liquid crystal display (LCD) on a hand held device, or other display device.

In one or more aspects of this disclosure, a non-transitory computer-readable medium stores programs for use by or in connection with an instruction execution system, apparatus, or device. More specific examples of a computer-readable medium may include by way of example and without limitation: a portable computer diskette, a random access memory (RAM), a read-only memory (ROM), an erasable programmable read-only memory (EPROM, EEPROM, or Flash memory), and a portable compact disc read-only memory (CDROM) (optical).

With further reference to FIG. 6, network interface device 206 may comprise various components used to transmit and/or receive data over a network environment. For example, the network interface 206 may include a device that can communicate with both inputs and outputs, for instance, a modulator/demodulator (e.g., a modem), wireless (e.g., radio frequency (RF)) transceiver, a telephonic interface, a bridge, a router, network card, etc.). The apparatus 1010 may communicate with one or more computing devices 103a, 103b (not shown) via the network interface 206 over the network 118 (not shown). The apparatus 1010 may further comprise mass storage 226. The peripheral 211 interface supports various interfaces including, but not limited to IEEE-1394 High Performance Serial Bus (Firewire), USB, a serial connection, and a parallel connection.

The apparatus 1010 shown in FIG. 6 may be embodied, for example, as a magnetic resonance apparatus, which includes a processing module or logic for performing conditional data processing, and may be implemented either off-line or directly in a magnetic resonance apparatus. For such embodiments, the apparatus 1010 may be implemented as a multi-channel, multi-coil system with advanced parallel image processing capabilities, and direct implementation makes it possible to generate images, for example, immediate T1 maps, available for viewing immediately after image acquisition, thereby allowing re-acquisition on-the-spot if necessary. As noted above, however, the apparatus 1010 need not be limited to a magnetic resonance imaging apparatus, but may include ultrasound and CT imaging apparatus and any other quantitative imaging apparatus characterized by consistency of image contrast either within or between images.

Although the programming logic, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowchart of FIG. 1 shows an example of functionality that may be implemented in the apparatus 1010 of FIG. 6. If embodied in software, each block shown in FIG. 1 may represent a module, segment, or portion of code that comprises program instructions to implement the specified logical function(s). The program instructions may be embodied in the form of source code that comprises machine code that comprises numerical instructions recognizable by a suitable execution system such as the processing device 202 (FIG. 6) in a computer system or other system. The machine code may be converted from the source code, etc. If embodied in hardware, each block may represent a circuit or a number of interconnected circuits to implement the specified logical function(s). Where any component discussed herein is implemented in the form of software, any one or more of a number of programming languages may be employed such as, for example, C, C++, C#, Objective C, Java®, JavaScript®, Perl, PHP, Visual Basic®, Python®, Ruby, Flash®, or other programming languages.

Although the flowchart of FIG. 1 shows a specific order of execution, it is understood that the order of execution may differ from that which is depicted. For example, the order of execution of two or more blocks may be scrambled relative to the order shown. Also, two or more blocks shown in succession in FIG. 1 may be executed concurrently or with partial concurrence. Further, in some embodiments, one or more of the blocks shown in FIG. 1 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein that comprises software or code can be embodied in any non-transitory computer-readable medium for use by or in connection with an instruction execution system such as, for example, a processing device 202 in a computer system or other system. In this sense, each may comprise, for example, statements including instructions and declarations that can be fetched from the computer-readable medium and executed by the instruction execution system.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed:

1. A method for providing a color map for medical imaging, comprising the steps of:
(a) acquiring a medical image;
(b) generating a color lookup table for representation of the medical image by:
(i) assigning a specific color to represent an established normal range of values in the image using one or more statistical descriptors of respective data distributions,
(ii) assigning the extent of deviation from the normal color or from an accepted threshold using a statistical descriptor to distinguish an altered state,
(iii) using colors different than the color assigned in step b(i) to represent targets for the deviation from the normal range of values or from accepted thresholds or additional normal ranges for one or more additional types of tissues, and
(iv) setting color transitions applying the respective statistical descriptors as in step b(ii); and
(c) applying the generated color lookup table for the color transitions to the medical image.

2. The method of claim 1, wherein the step of acquiring a medical image includes acquiring information on the distribution of image intensities for a tissue of a selected type of on the medical image.

3. The method of claim 1, wherein the colors used to represent targets in step b(iii) are assigned using one or more statistical descriptors as in step b(i).

4. The method of claim 1, wherein the altered state is a disease or an intervention state.

5. The method of claim 4, wherein the step of assigning the extent of deviation includes use of respective standard deviations, standard errors, percentile ranges or detection thresholds for desired sensitivity or specificity of the respective altered state detection.

6. The method of claim 1, wherein the step of using colors different that the color assigned in step b(i) includes using accepted contrasting colors to represent targets for the deviation from the normal range of values or from accepted thresholds.

7. The method of claim 6, wherein the step of using colors different than the color assigned in step b(i) includes using the descriptor in step b(ii).

8. The method of claim 1, wherein the one or more statistical descriptors are selected from the group consisting of mean (average) value, standard deviation (SD), standard error of mean (SEM), Confidence ranges, median and/or percentile ranges with any respective sums, and multiplications.

9. The method of claim 1, wherein the one or more statistical descriptor is calculated from prior studies or calculated for each dataset separately by selecting adequate reference areas.

10. The method of claim 1, wherein other formal statistical descriptors that can be determined with respect to established differences from abnormal conditions are used.

11. The method of claim 10, wherein the other formal statistical descriptors involve sensitivity or specificity based thresholds.

12. The method of claim 1, wherein the medical imaging includes quantitative medical imaging.

13. The method of claim 12, wherein the medical imaging is selected from the group consisting of magnetic resonance (MR) imaging (including but not limited to T1 mapping), ultrasound or computed tomography (CT) and the method is used to replace traditional grayscale.

14. The method of claim 12, wherein the medical imaging is magnetic resonance (MR) imaging T1 mapping.

15. The method of claim 1, further including the step of performing the method for any number of tissue classes, or any number of statistical descriptors to generate one or color maps to suit desired extent of clinical diagnostic sensitivity or specificity.

16. The method of claim 1, further including the step of representing one or more target tissue classes in a single image, with a deviation from norm being represented for each tissue class in either the same or different color transitions.

17. The method of claim 1, wherein the deviation is derived from population data statistics.

18. The method of claim 1, wherein the color transitions use a non-linear function.

19. The method of claim 1, wherein the transitions involve manipulation of at least one of the color, line, saturation or brightness of the image.
